(19) United States
(12) Patent Application Publication
(10) Pub. No.: US 2023/0158144 A1
(43) Pub. Date: May 25, 2023

(54) ULTRASOUND-TRIGGERED NANOCARRIERS

(71) Applicant: UNIVERSITY OF UTAH RESEARCH FOUNDATION, Salt Lake City, UT (US)

(72) Inventors: Jan Kubanek, Salt Lake City, UT (US); Natalya Y. Rapoport, Salt Lake City, UT (US)

(21) Appl. No.: 17/993,693

(22) Filed: Nov. 23, 2022

Related U.S. Application Data
(60) Provisional application No. 63/283,110, filed on Nov. 24, 2021.

(51) Int. Cl. A61K 41/00; A61K 9/51; A61K 31/05; A61K 31/5377; A61K 31/135; A61K 49/00

(52) U.S. Cl. CPC A61K 41/0047; A61K 9/5123; A61K 31/05; A61K 31/5377; A61K 31/135; A61K 9/5153; A61K 49/0002

(57) ABSTRACT

Described herein are high-boiling-point-based nanoparticles that release drugs specifically at the focus of ultrasound. The specific conjunction of the high-boiling-point-based nanoparticle formulation with low-frequency ultrasound can be used to deliver therapeutics in a safe and effective manner. The effectiveness and safety of the release is validated in vitro and in non-human primates.

### CROSS-REFERENCE TO RELATED APPLICATIONS

This application claims priority to U.S. Provisional Patent Application No. 63/283,110 filed on Nov. 24, 2021, which is incorporated by reference herein in its entirety.

### FEDERALLY SPONSORED RESEARCH

This invention was made with government support under NS100986 awarded by the National Institutes of Health. The government has certain rights in the invention.

### TECHNICAL FIELD

Described herein are high-boiling-point-based nanoparticles that can be activated with ultrasound. The specific conjunction of nanoparticle formulation and ultrasound parameters can be used to deliver therapeutics in an effective and safe manner.

### BACKGROUND

Treatments of mental, neurological, and other disorders are often curbed by intolerable side effects or low effectiveness of drugs. Consequently, millions of patients remain resistant to treatments and suffer from poor quality of life. There is a critical need for approaches that deliver medication selectively in the desired targets and at a high concentration.

Pioneering work in this domain devised temperature-sensitive liposomes that can be activated by heat or radiation. However, localized heating at depth has been challenging to achieve safely and in a controlled manner. Recent efforts have shifted to using ultrasound as a safe and practical form of energy for localized drug release. Ultrasound can be controllably focused at depth into specific tissue targets using inexpensive hardware. Targeting systemically injected drug carriers, ultrasound can trigger focal release with minimal off-target effects (FIG. 1).

Several groups have shown that ultrasound can trigger drug release from nano-sized structures stabilized with biocompatible shells. These nano-sized structures have been commonly filled with perfluorocarbon (PFC) cores. PFCs are highly inert and bestow the resulting nanoparticles with sensitivity to ultrasound. When exposed to ultrasound, the PFC core has been hypothesized to change phase from liquid to gas, greatly expanding in volume, and thus mediating drug release. Harnessing this understanding, a majority of previous studies used nanoparticles with PFC boiling points below body temperature. From a safety perspective, however, the use of PFCs with higher boiling point is preferable to reduce the risk of embolism. For instance, FDA approvals have been readily granted to PFCs with high boiling points to be administered into humans in large quantities. Nonetheless, drug-release from such nanoparticles has appeared to be less effective.

What is needed are high-boiling-point-based nanoparticles that can be activated with ultrasound to release active pharmaceutical agents. The specific conjuction of nanoparticle formulation and ultrasound parameters could provide treatments that are both effective and safe.

### SUMMARY

One embodiment described herein is an ultrasound release-inducible nanoparticle composition, comprising: a nanoparticle core comprising perfluorooctylbromide (PFOB); a copolymer matrix shell surrounding the nanoparticle core; and one or more pharmaceutical agents. In one aspect, the one or more pharmaceutical agents comprise propofol, ketamine, or mycophenolate mofetil. In another aspect, the copolymer matrix shell comprises poly(ethylene glycol)-β-poly(D,L-lactide) (PEG:PDLLA) block copolymer. In another aspect, the ultrasound release-inducible nanoparticle composition is configured to release the one or more pharmaceutical agents from the nanoparticle composition upon application of ultrasound. In another aspect, the ultrasound frequency comprises about 100 kHz to about 650 kHz. In another aspect, the ultrasound frequency comprises about 300 kHz. In another aspect, the ultrasound release-inducible nanoparticle composition is configured to release the one or more pharmaceutical agents from the nanoparticle composition upon application of an ultrasound pressure ranging from about 1 MPa to about 3 MPa. In another aspect, the ultrasound pressure comprises about 2 MPa. In another aspect, the ultrasound release-inducible nanoparticle composition has a boiling point of up to about 142° C. In another aspect, the ultrasound release-inducible nanoparticle composition has a diameter size ranging from about 300 nm to about 900 nm.

Another embodiment described herein is a method of releasing or activating one or more pharmaceutical agents from an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; and administering ultrasound to the ultrasound release-inducible nanoparticle composition to release or activate the one or more pharmaceutical agents. In one aspect, the one or more pharmaceutical agents comprise propofol, ketamine, or mycophenolate mofetil. In another aspect, the copolymer matrix shell comprises poly(ethylene glycol)-p-poly(D,L-lactide) (PEG:PDLLA) block copolymer. In another aspect, the ultrasound frequency comprises about 200 kHz to about 400 kHz. In another aspect, the ultrasound frequency comprises about 300 kHz.

Another embodiment described herein is a method of treating a subject suffering from a disease or disorder using an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; administering the ultrasound release-inducible nanoparticle composition to the subject; and administering ultrasound to the subject to release the one or more pharmaceutical agents from the ultrasound release-inducible nanoparticle composition. In one aspect, the one or more pharmaceutical agents comprise propofol, ketamine, or mycophenolate mofetil. In another aspect, the copolymer matrix shell comprises poly(ethylene glycol)-β-poly(D,L-lactide) (PEG:PDLLA) block copolymer. In another aspect, the ultrasound frequency comprises about 200 kHz to about 400 kHz. In another aspect, the low ultrasound frequency comprises about 300 kHz.

Another embodiment described herein is a method of performing imaging in a subject using an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; administering the ultrasound release-inducible nanoparticle composition to the subject; administering ultrasound to the subject to release the one or more pharmaceutical agents from the ultrasound release-inducible nanoparticle composition; and performing imaging on the subject. In one aspect, the imaging comprises CT, MRI, ultrasound, or combinations thereof. In another aspect, the one or more pharmaceutical agents comprise propofol, ketamine, or mycophenolate mofetil.

Another embodiment described herein is the use of an ultrasound release-inducible nanoparticle composition for administering one or more pharmaceutical agents to a subject suffering from a disease or disorder, the nanoparticle composition comprising: a nanoparticle core comprising perfluorooctylbromide (PFOB); a copolymer matrix shell surrounding the nanoparticle core; and one or more pharmaceutical agents. In one aspect, the one or more pharmaceutical agents is administered to the subject by a method comprising: administering the ultrasound release-inducible nanoparticle composition to the subject; and administering ultrasound to the subject to release the one or more pharmaceutical agents from the ultrasound release-inducible nanoparticle composition. In another aspect, the one or more pharmaceutical agents comprise propofol, ketamine, or mycophenolate mofetil.

Another embodiment described herein is the use of an ultrasound release-inducible nanoparticle composition for performing imaging in a subject, the nanoparticle composition comprising: a nanoparticle core comprising perfluorooctylbromide (PFOB); a copolymer matrix shell surrounding the nanoparticle core; and one or more pharmaceutical agents. In one aspect, the imaging comprises CT, MRI, ultrasound, or combinations thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows controlled drug release in a specific body location. Drug-filled biodegradable nanoparticles are injected into the bloodstream. Brief pulses of focused, low-intensity ultrasound are applied from the outside of the body using a standard transducer. At the ultrasound target, the focused pressure wave mechanically stimulates the nanoparticles, which leads to a release of the drug specifically at the target and not elsewhere.

FIG. 2A-B show study design and predictions. FIG. 2A shows the release should increase/decrease with the ultrasound frequency under a thermal or mechanical effect. FIG. 2B shows thermal effect, such as the hypothesized phase change, should be accentuated by PFCs with lower boiling points.

FIG. 3A-B show release from nanoparticles with distinct cores under two ultrasound frequency modes. Mean±S.E.M. propofol release relative to the amount encapsulated for the two ultrasound frequencies (FIG. 3A, combined across all cores) and the three cores (FIG. 3B) tested. The ultrasound was delivered in 100 ms pulses repeated 60 times over the period of 1 minute. The p-value denotes the significance of a two-sample two-sided t-test. A complete statistical analysis of the effects is provided in Table 1.

FIG. 4A-B show release across all tested factors. Mean±S.E.M. percentage of the released propofol for the two ultrasound frequencies. FIG. 4A, shows 300 kHz and FIG. 4B shows, 900 kHz ultrasound pressure amplitude (abscissa), and the three cores tested. n=3 samples for each core and ultrasound parameter except 0 MPa, where n=4. The thick lines represent quadratic fits to the data. Notably, for the 300 kHz data, linear fits were as explanatory as the quadratic fits.

FIG. 5A-B show stability of PFOB and PFP-based nanoparticles over time. FIG. 5A shows mean±S.E.M. diameter measured at the times as indicated on the abscissa. The times were measured relative to the time of the completion of the nanoparticle production. The data include n=6 samples of PFOB nanoparticles and n=6 samples of PFP nanoparticles. The error bars for PFOB are smaller than the symbols. FIG. 5B shows PFP and PFOB nanoparticle average size change after one hour at 22° C. or 37° C. There was no significant difference in either case.

FIG. 6 shows safety of PFOB-based nanoparticles. The heart rates of two macaque monkeys, one marmoset monkey, and three rats before and after intravenous injections of PFOB-based nanoparticles. The nanoparticles were filled with propofol at a concentration of 1 mg/kg (macaques), 1 mg/kg (marmoset), and 0.5, 1, and 1 mg/kg (rats). The injected volumes were 5 mL (macaque 1), 10 mL (macaque 2), 2 mL (marmoset), and 0.48, 0.53, and 1.1 mL, respectively (rats). In the marmoset, two injections were performed separated by 45 minutes. The EKG was recorded using a portable EKG-monitoring system. The heart rate in the smaller animals (marmoset and rats) tracked the frequently varying levels of isoflurane anesthesia—higher levels led to lower heart rate and reversely.

FIG. 7 shows blood clearance kinetics in macaque monkeys. Relative fluorescence as a function of specific sampling times indicated on the abscissa. The PFOB-based nanoparticles contained propofol and an infrared dye (IR800RS, LI-COR). The nanoparticles were injected via a saphenous vein catheter at time 0 at a volume of 5 mL. Blood samples (1 mL each) were taken at the times indicated on the abscissa and subjected to fluorescence analysis. The data points were captured using the superposition of two exponentials. One showed a fast and the other a slow time constant (see inset).

FIG. 8 shows biodegradation in major organs. Distribution of PFOB-based nanoparticles in major organs of the marmoset and the rats. The presence was assessed using the same infrared dye as in FIG. 7. The figure shows the percentage of total fluorescence (and the relative accumulation of the nanoparticles) measured within the respective organs. The rat data are presented as means±standard deviation.

FIG. 9 shows an outline of the production process for the nanoparticles. The conversion of polymeric micelles into PFC-core-based nanoparticles is achieved using ultrasound. FIG. 10A-B shows effective and safe release of propofol within deep brain targets of non-human primates. FIG. 10A shows nanoparticle administration. The nanoparticles are introduced into the circulation of awake animals using vascular access ports. FIG. 10B shows the effectiveness of the release. Propofol-filled PFOB nanoparticles (propofol concentration of 0.5 mg/kg) induce ipsilateral bias in the choice behavior. Immediately following the injection (green bar), low-intensity ultrasound targeted either the right (orange) or the left (blue) lateral geniculate nucleus (LGN). The black bars indicate the time epochs during which the difference in the propofol-induced effects was significant (two-tailed t-test).

FIG. 11 shows the release of three different active pharmaceutical agents, propofol, mycophenolate motefil, and ketamine, from nanoparticles as a function of ultrasound pressure.

Propofol and mycophenolate motefil release were analyzed using UV/visible spectroscopy. Ketamine release was measured using UV/visible fluorescence.

FIG. 12A-B show nanoparticles imaged using computerized tomography (CT). FIG. 12A shows nanoparticles containing PFOB have contrast under CT. FIG. 12B shows nanoparticles without PFOB are not visible under CT.

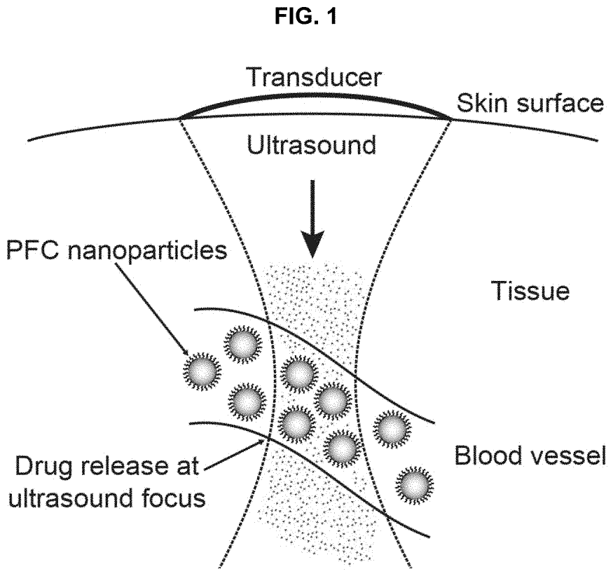

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Selective delivery of medication into specified tissue targets would realize the promise of personalized medicine with minimal side effects. Focused ultrasound provides noninvasive and practical means to release drugs from nanocarriers selectively at its target. However, which nanocarrier formulations provide safe and effective release and under which ultrasound parameters has been unclear. To expedite regulatory approval, the release effectiveness from nanocarriers containing perfluorocarbon cores of relatively high boiling points (up to 142° C.) was tested. Nanoparticles with these high boiling point perfluorocarbon cores show excellent stability compared with lower boiling points cores used previously. The safety of these nanocarriers was confirmed and their clearance kinetics and organ biodegradation characterized in non-human primates. Crucially, it was found that these safe, high-boiling-point nanocarriers can be used for effective release so long as they are activated by low-frequency ultrasound (100-650 kHz). This study informs the formulation and release parameters for safe and effective drug delivery in specific parts of the body or brain regions.

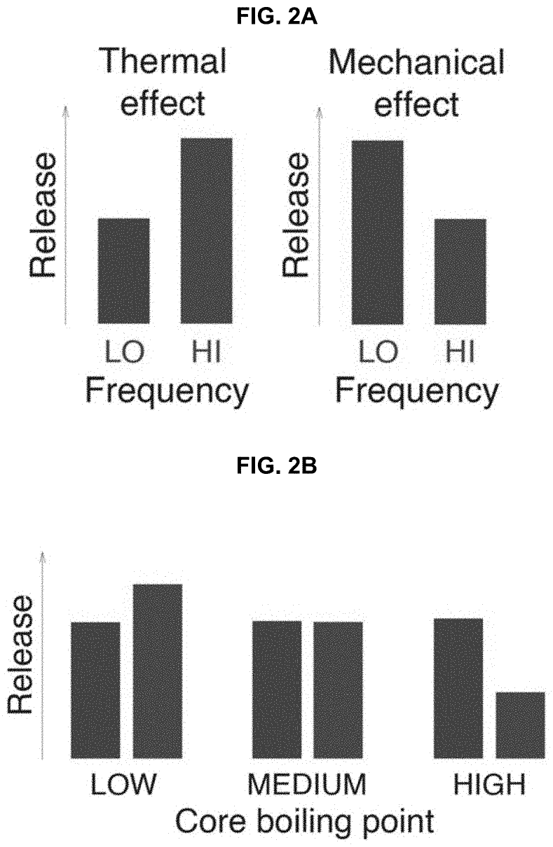

This study was based on two hypotheses. First, drug release from PFC-based nanoparticles may involve mechanical or thermal effects. The ultrasound frequency can engage these two mechanisms differentially, with high frequencies more likely to engage thermal mechanisms by depositing more energy at the target. Low frequencies are more likely to induce mechanical effects including particle displacement, cavitation, or vaporization of dissolved gases in the PFC core (FIG. 2A). Second, according to the predominant hypothesis of release, ultrasound delivered at the target induces vaporization of the PFC core. This implies that the PFC boiling point should be inversely related to release effectiveness and that this effect will be observed at high ultrasound frequencies (FIG. 2B).

One embodiment described herein is an ultrasound release-inducible nanoparticle composition, comprising: a nanoparticle core comprising perfluorooctylbromide (PFOB); a copolymer matrix shell surrounding the nanoparticle core; and one or more pharmaceutical agents. In one aspect, the one or more pharmaceutical agents comprise propofol, ketamine, or mycophenolate mofetil. In another aspect, the copolymer matrix shell comprises poly(ethylene glycol)-β-poly(D,L-lactide) (PEG:PDLLA) block copolymer. In another aspect, the ultrasound release-inducible nanoparticle composition is configured to release the one or more pharmaceutical agents from the nanoparticle composition upon application of ultrasound. In another aspect, the ultrasound frequency comprises about 100 kHz to about 650 kHz. In another aspect, the ultrasound frequency comprises about 300 kHz. In another aspect, the ultrasound release-inducible nanoparticle composition is configured to release the one or more pharmaceutical agents from the nanoparticle composition upon application of an ultrasound pressure ranging from about 1 MPa to about 3 MPa. In another aspect, the ultrasound pressure comprises about 2 MPa. In another aspect, the ultrasound release-inducible nanoparticle composition has a boiling point of up to about 142° C. In another aspect, the ultrasound release-inducible nanoparticle composition has a diameter size ranging from about 300 nm to about 900 nm.

Another embodiment described herein is a method of releasing or activating one or more pharmaceutical agents from an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; and administering ultrasound to the ultrasound release-inducible nanoparticle composition to release or activate the one or more pharmaceutical agents. In one aspect, the one or more pharmaceutical agents comprise propofol, ketamine, or mycophenolate mofetil. In another aspect, the copolymer matrix shell comprises poly(ethylene glycol)-β-poly(D,L-lactide) (PEG:PDLLA) block copolymer. In another aspect, the ultrasound frequency comprises about 200 kHz to about 400 kHz. In another aspect, the ultrasound frequency comprises about 300 kHz.

Another embodiment described herein is a method of treating a subject suffering from a disease or disorder using an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; administering the ultrasound release-inducible nanoparticle composition to the subject; and administering ultrasound to the subject to release the one or more pharmaceutical agents from the ultrasound release-inducible nanoparticle composition. In one aspect, the one or more pharmaceutical agents comprise propofol, ketamine, or mycophenolate mofetil. In another aspect, the copolymer matrix shell comprises poly(ethylene glycol)-β-poly(D,L-lactide) (PEG:PDLLA) block copolymer. In another aspect, the ultrasound frequency comprises about 200 kHz to about 400 kHz. In another aspect, the low ultrasound frequency comprises about 300 kHz.

Another embodiment described herein is a method of performing imaging in a subject using an ultrasound release-inducible nanoparticle composition, the method comprising: preparing an ultrasound release-inducible nanoparticle composition comprising: a nanoparticle core comprising perfluorooctylbromide (PFOB); a copolymer matrix shell surrounding the nanoparticle core; and one or more pharmaceutical agents; administering the ultrasound release-inducible nanoparticle composition to the subject; administering ultrasound to the subject to release the one or more pharmaceutical agents from the ultrasound release-inducible nanoparticle composition; and performing imaging on the subject. In one aspect, the imaging comprises CT, MRI, ultrasound, or combinations thereof. In another aspect, the one or more pharmaceutical agents comprise propofol, ketamine, or mycophenolate mofetil.

Another embodiment described herein is the use of an ultrasound release-inducible nanoparticle composition for administering one or more pharmaceutical agents to a subject suffering from a disease or disorder, the nanoparticle composition comprising: a nanoparticle core comprising perfluorooctylbromide (PFOB); a copolymer matrix shell surrounding the nanoparticle core; and one or more pharmaceutical agents. In one aspect, the one or more pharmaceutical agents is administered to the subject by a method comprising: administering the ultrasound release-inducible nanoparticle composition to the subject; and administering ultrasound to the subject to release the one or more pharmaceutical agents from the ultrasound release-inducible nanoparticle composition. In another aspect, the one or more pharmaceutical agents comprise propofol, ketamine, or mycophenolate mofetil

Another embodiment described herein is the use of an ultrasound release-inducible nanoparticle composition for performing imaging in a subject, the nanoparticle composition comprising: a nanoparticle core comprising perfluorooctylbromide (PFOB); a copolymer matrix shell surrounding the nanoparticle core; and one or more pharmaceutical agents. In one aspect, the imaging comprises CT, MRI, ultrasound, or combinations thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. An ultrasound release-inducible nanoparticle composition, comprising:
a nanoparticle core comprising perfluorooctylbromide (PFOB);
a copolymer matrix shell surrounding the nanoparticle core; and
one or more pharmaceutical agents.

Clause 2. The composition of clause 1, wherein the one or more pharmaceutical agents comprise propofol, ketamine, or mycophenolate mofetil.

Clause 3. The composition of clause 1 or 2, wherein the copolymer matrix shell comprises poly(ethylene glycol)-β-poly(D,L-lactide) (PEG:PDLLA) block copolymer.

Clause 4. The composition of any one of clauses 1-3, wherein the ultrasound release-inducible nanoparticle composition is configured to release the one or more pharmaceutical agents from the nanoparticle composition upon application of ultrasound.

Clause 5. The composition of any one of clauses 1-4, wherein the ultrasound frequency comprises about 100 kHz to about 650 kHz.

Clause 6. The composition of any one of clauses 1-5, wherein the ultrasound frequency comprises about 300 kHz.

Clause 7. The composition of any one of clauses 1-6, wherein the ultrasound release-inducible nanoparticle composition is configured to release the one or more pharmaceutical agents from the nanoparticle composition upon application of an ultrasound pressure ranging from about 1 MPa to about 3 MPa.

Clause 8. The composition of any one of clauses 1-7, wherein the ultrasound pressure comprises about 2 MPa.

Clause 9. The composition of any one of clauses 1-8, wherein the ultrasound release-inducible nanoparticle composition has a boiling point of up to about 142° C.

Clause 10. The composition of any one of clauses 1-9, wherein the ultrasound release-inducible nanoparticle composition has a diameter size ranging from about 300 nm to about 900 nm.

Clause 11. A method of releasing or activating one or more pharmaceutical agents from an ultrasound release-inducible nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents; and
administering ultrasound to the ultrasound release-inducible nanoparticle composition to release or activate the one or more pharmaceutical agents.

Clause 12. The method of clause 11, wherein the one or more pharmaceutical agents comprise propofol, ketamine, or mycophenolate mofetil.

Clause 13. The method of clause 11 or 12, wherein the copolymer matrix shell comprises poly(ethylene glycol)-β-poly(D,L-lactide) (PEG:PDLLA) block copolymer.

Clause 14. The method of any one of clauses 11-13, wherein the ultrasound frequency comprises about 100 kHz to about 650 kHz.

Clause 15. The method of any one of clauses 11-14, wherein the low intensity ultrasound frequency comprises about 300 kHz.

Clause 16. A method of treating a subject suffering from a disease or disorder using an
ultrasound release-inducible nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents;
administering the ultrasound release-inducible nanoparticle composition to the subject; and
administering ultrasound to the subject to release the one or more pharmaceutical agents from the ultrasound release-inducible nanoparticle composition.

Clause 17. The method of clause 16, wherein the one or more pharmaceutical agents comprise propofol, ketamine, or mycophenolate mofetil. Clause 18. The method of clause 16 or 17, wherein the copolymer matrix shell comprises poly(ethylene glycol)-β-poly(D,L-lactide) (PEG:PDLLA) block copolymer.

Clause 19. The method of any one of clauses 16-18, wherein the ultrasound frequency comprises about 100 kHz to about 650 kHz.

Clause 20. The method of any one of clauses 16-19, wherein the low intensity ultrasound frequency comprises about 300 kHz.

Clause 21. A method of performing imaging in a subject using an ultrasound release-inducible
nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents;
administering the ultrasound release-inducible nanoparticle composition to the subject;
administering ultrasound to the subject to release the one or more pharmaceutical agents from the ultrasound release-inducible nanoparticle composition; and performing imaging on the subject.

Clause 22. The method of clause 21, wherein the imaging comprises CT, MRI, ultrasound, or combinations thereof.

Clause 23. The method of clause 21 or 22, wherein the one or more pharmaceutical agents comprise propofol, ketamine, or mycophenolate mofetil.

Clause 24. Use of an ultrasound release-inducible nanoparticle composition for administering
one or more pharmaceutical agents to a subject suffering from a disease or disorder, the
nanoparticle composition comprising:
a nanoparticle core comprising perfluorooctylbromide (PFOB);
a copolymer matrix shell surrounding the nanoparticle core; and
one or more pharmaceutical agents.

Clause 25. The use of clause 24, wherein the one or more pharmaceutical agents is
administered to the subject by a method comprising:
administering the ultrasound release-inducible nanoparticle composition to the subject; and
administering ultrasound to the subject to release the one or more pharmaceutical agents from the ultrasound release-inducible nanoparticle composition.

Clause 26. The use of clause 24 or 25, wherein the one or more pharmaceutical agents comprise propofol, ketamine, or mycophenolate mofetil.

Clause 27. Use of an ultrasound release-inducible nanoparticle composition for performing
imaging in a subject, the nanoparticle composition comprising:
a nanoparticle core comprising perfluorooctylbromide (PFOB);
a copolymer matrix shell surrounding the nanoparticle core; and
one or more pharmaceutical agents.

Clause 28. The use of clause 27, wherein the imaging comprises CT, MRI, ultrasound, or combinations thereof.

### EXAMPLES

### Example 1

### Materials and Methods

Methoxy poly(ethylene glycol)-β-poly(D,L-lactide) (PEG-PDLLA) co-polymers with 2,000:2,200 g/mol molecular weights, respectively, were obtained from PolyScitech (USA). 2H,3H-decafluoropentane and perfluorooctyl bromide were obtained from Tokyo Chemical Industry Co. (Japan). Perfluoro-n-pentane was obtained from Strem Chemicals (USA). Propofol was obtained from Sigma Aldrich (Millipore Sigma, Canada). Infrared dye IR800RS NHS Ester was obtained from LI-COR Biosciences (USA). HPLC-grade tetrahydrofuran (THF) and methanol were obtained from Fisher Scientific (USA). Phosphate buffer solution (PBS) was obtained from Gibco (Thermo Fisher Scientific, USA).

### Nanoparticle Production

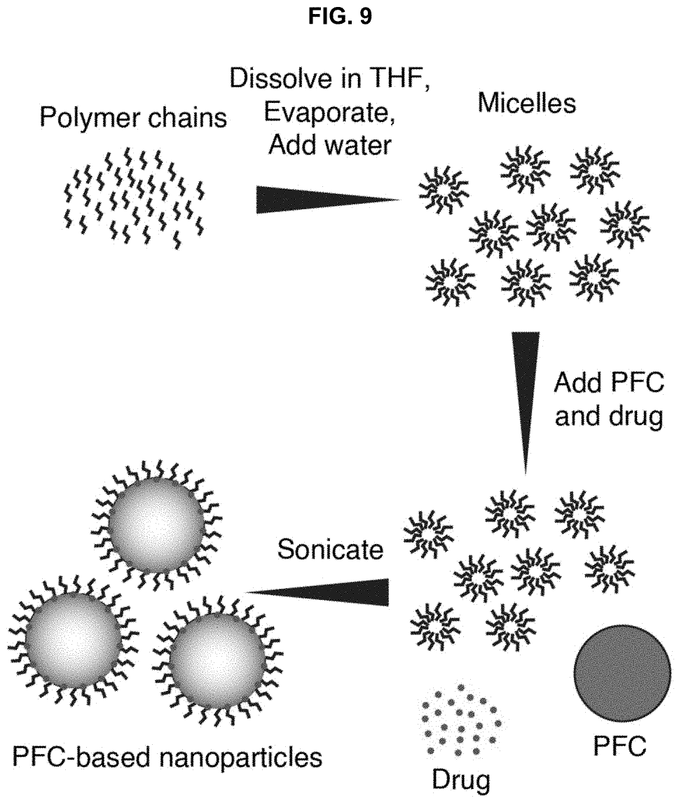

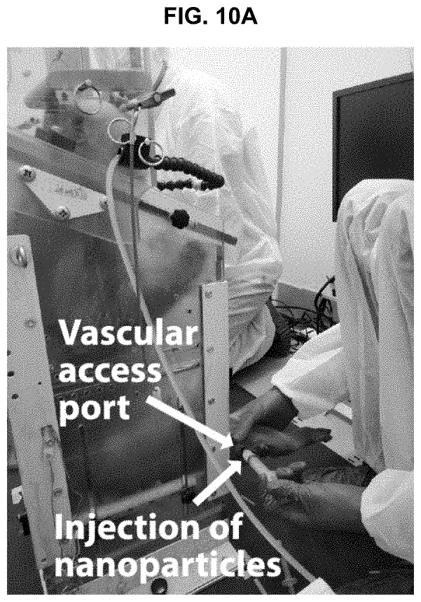

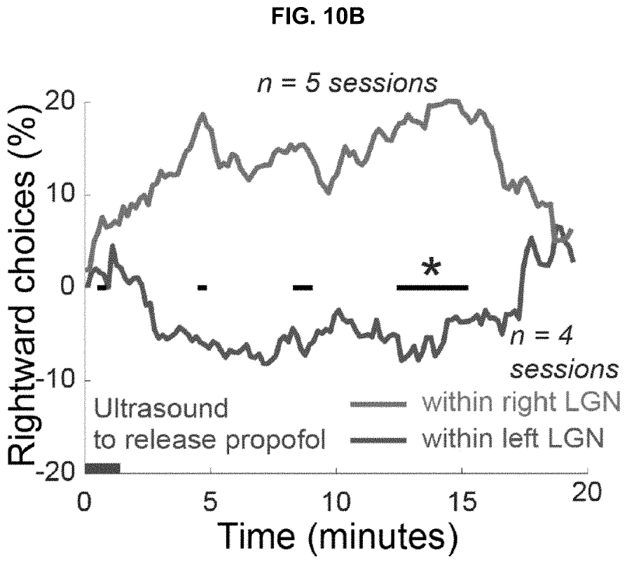

The process of manufacturing the drug-encapsulating, ultrasound-responsive PFC particles is illustrated at a conceptual level in (FIG. 9). The process converts small (<30 nm) micelles into much larger (>300 nm) PFC-filled nanoparticles. First, the PEG-PDLLA polymer constituting the basis of the nanoparticle shell is dissolved in THF at a rate of 1 mL THF: 16 mg polymer. For the biodistribution and blood clearance studies, infrared dye is added at a ratio of 1:32 (dye:polymer) for the rats and marmoset and 1:110 or 1:89 for the macaques 1 and 2, respectively. THF is then evaporated under vacuum until a gel-like layer remains. PBS is added at a rate of 1 mL PBS: 8 mg polymer and placed on a shaker table at 120 rpm to dissolve for 15 minutes. The addition of PBS orients the hydrophilic copolymer, PEG, toward the water and the hydrophobic, PDLLA, copolymer away from the water, and as a consequence, micelles are formed. Next, the PFC core and propofol are added and emulsified. A ratio of 1 mg propofol: 2 mg polymer was used in all cases. The nanoparticles' diameter can be controlled by the ratio of PFC to polymer, as reported previously. For PFOB and DFP nanoparticles, a ratio of 4.5 μL PFC: 1 mg polymer was used. The ratio for PFP was scaled up to 6.25 μL: 1 mg to account for PFC lost to vaporization before being emulsified. A 20 kHz, 500W sonicator with a cup horn attachment (VCX500, Sonics) was used to perturb the thermodynamic equilibrium of the micellar system, which leads to the incorporation of PFOB into the micelles and the formation of stable nanodroplets or nanoparticles. The PFC and propofol are added to 15 mL centrifuge tubes and gently shaken to combine before adding 8 mL of the micelle solution. The samples are then sonicated in a cold bath at 20% power in 30-second intervals until the solution is cloudy and drug and PFC are fully emulsified (between 60 and 90 seconds in total). A custom temperature-controlled cooling system maintained the bath temperature during sonication at 2° C. for PFP and 10° C. for DFP and PFOB. PFP must be kept colder to minimize vaporization before emulsification, while DFP and PFOB require higher temperatures to emulsify successfully. This controlled temperature approach maximizes the consistency of the nanoparticle sizes, drug encapsulation, and release properties. The resulting solution contains the desired nanoparticles in addition to remaining micelles, dissolved polymer, and free propofol. Nanoparticles are isolated using three cycles of centrifugation at 3,000 relative centrifugal force (RCF) at 4° C. After each cycle, the supernatant is discarded, and the pellet dissolved in 5 mL fresh PBS. If the resulting solution contains larger particles than needed, these were removed by a slower centrifuge cycle for 1 minute at 800 RCF, this time keeping the supernatant. Larger particles contained in the pellet are discarded. For animal experiments, the nanoparticle solutions were sterilized for 3 hours under UV light, a protocol previously shown to yield effective sterilization. Sterilization was conducted in glass vials in a custom chamber with an 8W UV lamp (Philips, USA).

### Nanoparticle Characterization

The sizes were measured using a Zetasizer Nano S (Malvern Panalytical, UK), which reports the intensity-weighted size distribution. The size values reported in the Results section describe the mean ±standard deviation of the distribution of the intensity values measured by the device. To quantify the amount of drug encapsulated, a 50 μL solution of nanoparticles is added to 450 μL of methanol to dissolve all components. A UV-Vis spectrophotometer (NanoDrop 2000, Thermo Scientific) is used to quantify the concentration by comparing the absorbance at 276 nm to a propofol standard curve.

### Apparatus

Drug release is quantified in standard 1.5 mL microcentrifuge tubes. Each tube with freshly produced nanoparticles is placed into a plastic holder. A focused ultrasonic transducer (H-115, 64 mm diameter, 52 mm focal depth, Sonic Concepts) was positioned 52 mm below the holder so the sample was within the ultrasound focus. Degassed water (AIMS III system with AQUAS-10 Water Conditioner, Onda) mediated coupling between the ultrasound face and the vial. The transducer was operated at 300 kHz and the third harmonic, 900 kHz. Stimuli were generated using a function generator (33520b, Keysight). The signals were amplified using a 55-dB, 300 kHz-30 MHz power amplifier (A150, Electronics & Innovation).

### Ultrasound Parameters

The ultrasound carrier frequencies for in vitro experiments were 300 kHz and 900 kHz. Continuous pulses 100 ms in duration were repeated once per second for a total of 60 seconds. The pressure levels at the vial location, measured in degassed water, were 0, 0.5, 0.9, 1.3, 1.7, 2.1, and 2.5 MPa. The pressure fields were measured using a capsule hydrophone (HGL-0200, Onda) calibrated between 250 kHz and 40 MHz and secured to 3-degree-of-freedom programmable translation system (Aims III, Onda).

For in vivo experiments, a similar ultrasound protocol was used to control for changes undergone by the particles as a result of sonication. The same transducer was operated at 248 kHz in 60-second blocks of 100 ms pulses. The animals were shaved, and the transducer was coupled to the scalp using a 2% agar cone and ultrasound gel. The maximum pressure was estimated to be 1 MPa for all animals. The pressure was estimated from free-field measurements using results from past studies, which indicate a transmission rate of 76% through the skull of a 780 g rat and 66% through a macaque skull. Transmission through the marmoset skull has been less thoroughly studied but is likely similar to rats since they are close to the same weight. For further efficacy studies, pressure at the focus should be more carefully estimated. Ultrasound was applied to the rats at the midline, 2 mm posterior to the eyes for 60 seconds 5 minutes after administration of nanoparticles. For the marmoset, ultrasound was applied at the posterior surface of the skull for 60 seconds 5 minutes after each of the two administrations. The right and left visual cortex were targeted independently for macaque 1. Ultrasound was applied in 60-second blocks over 90 minutes for a total of 4 sonication blocks per side starting 2 minutes after nanoparticle administration. Sonication of the left and right sides was interleaved. For macaque 2, the right and left visual cortex were targeted simultaneously using two ultrasound transducers to maximize the sonicated volume. In this macaque, sonications were repeater for only two 60-second blocks separated by two minutes.

### Drug Release Characterization

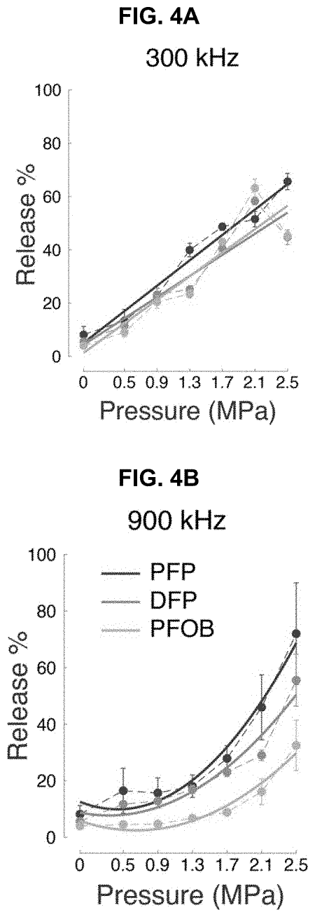

100 μL of hexane was placed on top of 200 μL nanoparticle solutions prior to sonication to function as a sink for released propofol. After 1 minute, 45 seconds of total incubation time, 50 μL of hexane was extracted. The amount of dissolved propofol was quantified using UV-Vis spectrophotometry as described previously. The percent release efficacy is defined as the amount of the drug released into the hexane relative to the amount encapsulated. Each datapoint in FIG. 4 included 3-4 distinct samples.

### Subjects

Two adult male old world monkeys (Macaca mulatta), an adult male new world monkey (Callithrix jacchus), and 3 adult male Sprague-Dawley rats participated in the safety and biodegradability experiments. All procedures were approved by the Institutional Animal Care and Use Committee of the University of Utah. The macaques weighed 10.8 and 16.1 kg, respectively. The rats weighed 900, 830, and 850 g. The marmoset weighed 288 g.

### Animal Procedures

For the vital sign experiments, the rats were anesthetized with 80 mg/kg ketamine and 10 mg/kg xylazine administered intraperitoneally. A dose of sterilized PFOB nanoparticles containing 0.5 mg/kg propofol was administered to the first animal and 1 mg/kg to the subsequent animals. A total of 0.48, 0.53, and 1.1 mL of the nanoparticle mixture was injected into the tail vein of the 3 animals. Biodistribution experiments were conducted one week later. The animals were anesthetized with 2.5-3% isoflurane, and dye-loaded nanoparticles were administered at a dose of 1 mg/kg propofol followed by an equal volume of sterile saline. After one hour, the animals were euthanized by coronary exsanguination under 5% isoflurane anesthesia.

The primates were preanesthetized with ketamine (25 mg/kg intramuscularly) and intubated with endotracheal tubes. They were artificially ventilated, and anesthesia maintained with 1-4% isoflurane throughout the procedure by veterinary staff. The animals were placed on warmed operating table pads to maintain body temperature. For the marmoset, dye-loaded nanoparticles were injected through the tail vein at doses of 1 mg/kg propofol for each of two injections separated by 45 minutes. A total volume of 2 mL of nanoparticle solution was administered, followed by an equal volume of sterile saline. The marmoset was euthanized by an overdose of sodium pentobarbital and perfused transcardially with 4% paraformaldehyde 82 minutes after the first injection. For macaque 1, one injection of dye-loaded nanoparticles was administered in the right saphenous vein at 1 mg/kg and a volume of 5 mL, followed by an equal volume of sterile saline. For macaque 2, the right and left cephalic veins were used with a 10 mL volume of dye-loaded nanoparticles. Blood samples were taken from the left saphenous vein 2, 10, 20, 40, 80, and 120 minutes, at a volume of 1 mL each. Following 120 minutes of monitoring, the macaques were euthanized by an overdose of sodium pentobarbital and perfused transcardially with 4% paraformaldehyde.

The procedures were painless and consistent with the recommendations of the Panel on Euthanasia of the American Veterinary Medical Association. In all cases, heart rate was recorded using a portable ECG-monitoring system with electrode pads placed on the upper right and upper left upper abdomen and a reference on the lower left abdomen.

### Quantification of Release Effectiveness and Nanoparticle Safety in Non-Human Primates

To quantify the effectiveness of the release, two non-human primates were implanted with vascular access ports. The ports enable the injection of nanoparticles into the circulation, akin to standard bolus in humans. The PFOB-based nanoparticles were filled with propofol at a concentration of 0.5 mg/kg and injected into the circulation of the awake subjects. The awake subjects participated in a visual task in which they decided whether a left or right visual target appeared first. They communicated their decision by making an eye movement to the chosen target. Low-intensity (1 MPa amplitude at target) and low-frequency ultrasound (450 kHz) were used to release the drug from the injected nanoparticles. The ultrasound targeted either the left or the right lateral geniculate nucleus (LGN), which is the primary relay of visual information into the brain. If an LGN is inhibited, animals are known to show ipsilateral bias in their visual choices. Indeed, the release of the neuroinhibitory drug propofol from the nanoparticle carriers caused ipsilateral bias in the animals' choices (FIG. 10B). Moreover, the effect was specific to the targeted site (left or right LGN corresponding to blue and orange plots in FIG. 10B, respectively). This effect confirms effective release of the drug in deep brain regions of non-human primates by focused ultrasound.

In the clinical blood tests, blood was drawn from the vascular access ports before and 1.5 hours after the administration of the nanoparticles. Standard clinical chemistry and hematology tests were performed (Table 2 provides a subset of the collected values).

### Measurements of Blood Clearance and Biodistribution

Following euthanasia, major organs were extracted and subjected to infrared fluorescence analysis using an IVIS system (Perkin Elmer). Excitation was set to 745 nm and emission to 820 nm, with 5 seconds of exposure. Extracted organs were placed directly on a dish in the field of view of the camera. In the macaques, the same organs as in the smaller animals were available, with the exception of the brain for macaque 1. Blood samples were pipetted into drops of 100 μL on a dish. Total fluorescence for each of these samples was quantified using Aura software (Spectral Instruments), defining regions of interest incorporating the whole organ or blood sample and subtracting a background region of the same size. Percent biodistribution was computed as the total fluorescence of the region containing the organ divided by the sum of the fluorescence of all organs. Nanoparticle concentration in the blood was computed as the amount of fluorescence from each sample relative to the first sample, obtained at 2 minutes.

### Example 2

### In Vitro Ultrasound-Triggered Drug Release

PFC-based, copolymer-stabilized nanoparticles were prepared using a similar approach as described in previous studies. The nanoparticles were loaded with the neuromodulatory drug propofol and quantified the effectiveness of its release using an approach described previously in which drug is released from the nanoparticles into an organic solvent. Uniquely, these experiments evaluated how the critical component of the nanoparticle—its core—governs the amount of drug released as a function of specific ultrasound parameters. Specifically, the release effectiveness of three different PFC cores—perfluoropentane (PFP), decafluoropentane (DFP), and perfluorooctylbromide (PFOB) was analyzed. These PFCs have boiling points of 29° C., 55° C., and 142° C., respectively. Nanoparticles with these cores had comparable sizes: mean±SD of 543.3±23.7, 550.8±91.7, and 473.0±28.4 nm for PFP, DFP, and PFOB-based nanoparticles, respectively.

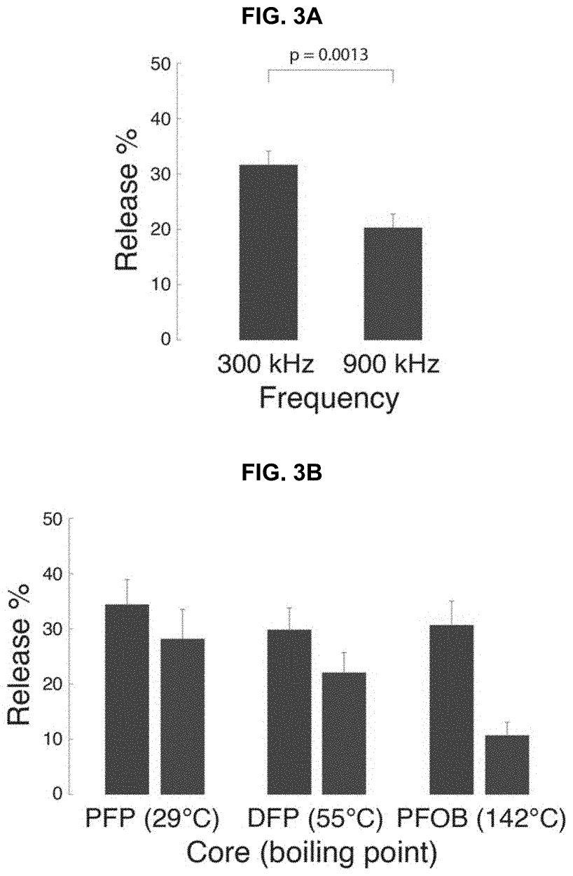

An ultrasonic transducer capable of operating at both low (300 kHz) and high (900 kHz) frequencies was focused on vials containing the nanoparticle solutions. Ultrasound of low frequency (300 kHz) triggered more drug release from the nanoparticles than ultrasound of high frequency (900 kHz) (FIG. 3A). The difference in the percentage of drug released at the two frequencies (31.7% and 20.3%, respectively) was significant when averaged across all cores and ultrasound pressures (t(130)=3.3, p=0.0013, two-sample two-tailed t-test). In line with the hypothesis, at the higher 900 kHz frequency, the release effectiveness strongly depends on the boiling point of the PFC core (FIG. 3B). The scaling is in the expected direction—the lower the boiling point of the PFC, the higher the release effectiveness. An omnibus ANOVA model (Table 1) that incorporated all factors evaluated (core, ultrasound frequency, ultrasound pressure) as well as all possible interactions, detected a significant core x frequency interaction (F(2, 90)=8.05, p=0.00061).

TABLE 1
Summary of the Effects. The effects of the nanoparticle
core, ultrasound frequency (F), and ultrasound pressure
(P). These effects were assessed using a three-way ANOVA
that featured the three main effects and all possible
interactions. Bold entries are significant (p < 0.05).

| Factor | Significance |
|---|---|
| Core | <0.001 |
| F | <0.001 |
| P | <0.001 |
| Core × F | <0.001 |
| F × P | <0.001 |
| Core × P | 0.030 |
| Core × F × P | 0.049 |

The scaling of the release effectiveness by the PFC boiling point (red bars in FIG. 3B) suggests the engagement of a thermal mechanism at the higher frequency, in agreement with previous propositions. If this is the case, the release should also scale with the average ultrasound intensity (I) delivered into the nanoparticles. For longitudinal waves, it holds I=P²/2Z, where P is the ultrasound pressure amplitude, and Z is the acoustic impedance of the medium. Thus, a thermal effect scales with pressure squared. The data at 900 kHz showed a quadratic dependence on pressure (FIG. 4B). Specifically, quadratic fits to the data (solid curves) explained 96.5, 93.5, and 94.8% of the variance in the PFP, DFP, and PFOB data points. In comparison, linear fits only explained 80.3, 80.5, and 69.3% of the variance, respectively. The difference in the mean variance explained by the quadratic and linear fits (94.9% versus 76.7%) was significant (t(2)=4.83, p=0.040, two-sided t-test). In contrast, the lower 300 kHz frequency showed a linear dependence of the release on pressure (FIG. 4, left). A quadratic fit did not significantly (p>0.16) differ from the linear fit in terms of variance explained (89.5% versus 89.3%). The linear dependence on pressure at 300 kHz is consistent with a mechanical rather than a thermal effect.

The effects of the three factors assessed (core, ultrasound frequency, and ultrasound pressure) as well as all possible interactions are summarized in an omnibus ANOVA model (Table 1). This analysis confirmed that both the core and ultrasound parameters (frequency and pressure) are important considerations for effective release. In addition, the factors interact: the selection of a specific core must be performed in conjunction with suitable ultrasound parameters.

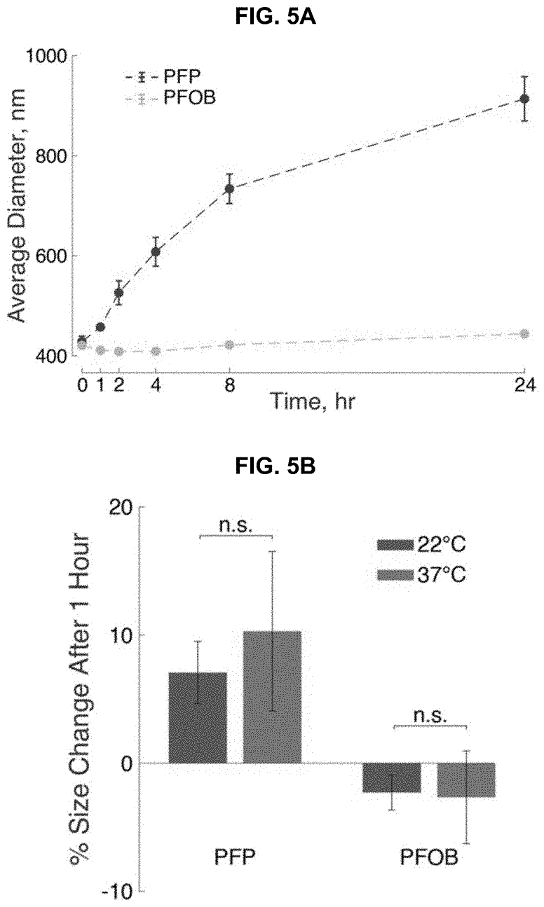

From the safety perspective, cores with higher boiling points can be expected to be more stable, thus minimizing the risk of embolism and spontaneous drug release when injected into the bloodstream. To quantify this notion, the sizes of PFP and PFOB nanoparticles were measured over the course of 24 hours (FIG. 5A). PFP-based nanoparticles more than doubled in size over the 24 hours, from mean±SD of 428±27 to 914±108 nm (t(5)=11.9, p<0.001, paired two-tailed t-test). In contrast, the size of PFOB-based nanoparticles remained steady, increasing only by 23 nm over the 24 hours (from 421±10 to 444±10 nm; t(5)=6.3, p=0.0015). A repeated measures ANOVA detected a highly significant interaction of PFC core and time (F(5, 50)=43.54, p<0.001), confirming that the PFP-based nanoparticles expanded at a higher rate than PFOB-based nanoparticles.

Additional experiments evaluated how the stability depends on temperature. Specifically, fresh PFP and PFOB nanoparticles were incubated at 37° C. for one hour. Temperature did not have a significant effect on the sizes of either PFP (t(10)=0.10, p=0.93, two-sided t-test) or PFOB (t(10)=−0.48, p=0.64) particles (FIG. 5B).

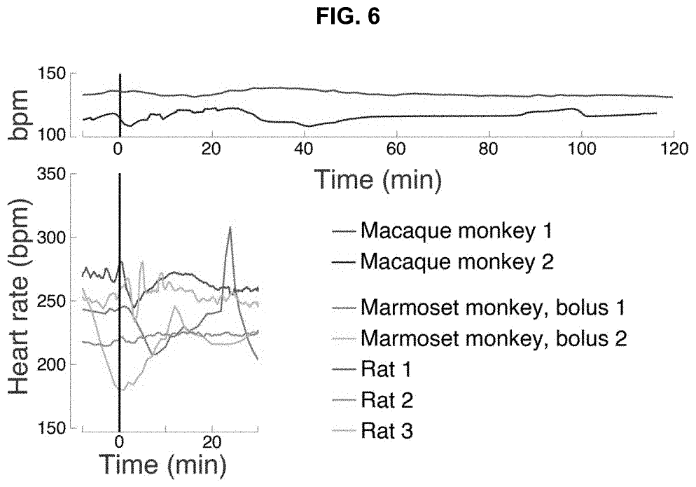

As demonstrated in FIG. 3B and FIG. 4A, the stable, PFOB-based nanoparticles can be as effective as the unstable PFP-based nanoparticles when driven at the lower, 300 kHz frequency. The safety, blood clearance kinetics, and organ biodistribution were assessed for the PFOB-based nanoparticles. These tests involved four macaque monkeys (two used during anesthesia (FIG. 6-8) and two during awake behavior (FIG. 10A-B)), a marmoset monkey, and 3 Sprague-Dawley rats.

### In Vivo Safety Studies

The injection of the propofol-filled PFOB nanoparticles into the bloodstream of the animals did not cause notable changes in vital signs. In particular, FIG. 6 shows the heart rate as a function of time, aligned to the time of the bolus administration. There was no significant difference between the heart rate measured during a 5-minute interval immediately prior to the bolus and the last 5 minutes of the measurements (203.4 compared with 204.5 beats per minute, t(5)=−0.19, p=0.85, paired two-sided t-test). The vital signs of the macaque monkeys were particularly steady.

The pharmacokinetics of the nanoparticles were investigated in the macaque monkeys.

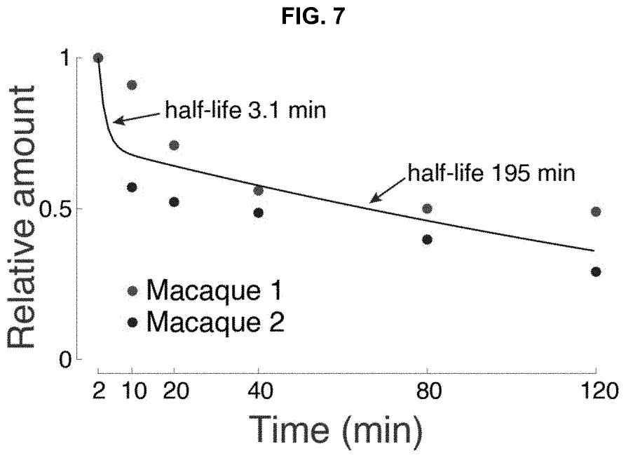

To do so, a fluorescent dye was incorporated in the nanoparticles along with propofol. While the macaques were anesthetized, blood samples were obtained at 2-, 10-, 20-, 40-, 80-, and 120-minutes following injection. The amount of fluorescence from these blood samples were quantitated relative to the 2-min time point (FIG. 7). This blood clearance curve has an initial half-life of 3.1 minutes followed by a slow decay with half-life 195 minutes, similar to previous reports with PFP-based nanoparticles in rats. An accurate fit to these data could only be made by superimposing two exponentials, one with a fast and one with a slow rate of decay (FIG. 7). This suggests that the clearance of the nanoparticles involved two distinct processes or organs.

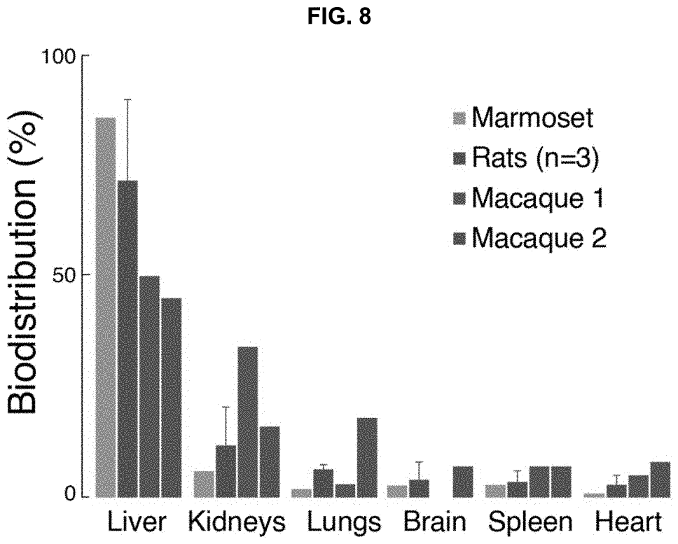

Finally, an assessment was made to determine in which organs the PFOB-based nanoparticles degrade. Immediately following the measurements of vital signs (FIG. 6), the animals were sacrificed, and their major organs extracted for analysis. The nanoparticles were found predominantly in the liver, in both the primates and rats (FIG. 8). The kidneys were the only other organs where average relative fluorescence exceeded 7%.

This study investigated the effectiveness and safety of drug release from biocompatible nanoparticles triggered by focused ultrasound. Uniquely, the release efficacy from nanoparticles filled with PFCs with distinct boiling points, including boiling points above the body temperature, were evaluated and the relevant ultrasound parameters that activate them were investigated. Low ultrasound frequencies (300 kHz in particular) are more effective in mediating release in general and that low frequencies are paramount for effective release from PFCs with a high boiling point. Moreover, these experiments validated the nanoparticles' safety and blood clearance kinetics with a high boiling point in non-human primates and noted no safety issues.

The core has been hypothesized to be a critical factor in governing the effectiveness of ultrasound-based drug release. Indeed, the 900 kHz data show (FIG. 3B) that at the range of commonly applied ultrasound frequencies 1 MHz), the core is a key determinant of release effectiveness. The higher the boiling point of the encapsulated PFC, the lower the release effectiveness. This finding is in accord with previous results. Lowering the frequency to 300 kHz increased the release effectiveness at a general, core-independent level (FIG. 3). The application of low-frequency ultrasound thus opens the path to using high-boiling-point cores (e.g., PFOB) as release actuators. A frequency of 300 kHz provides spatial resolution on the order of several millimeters, which approximates the focal volume of the drug released in tissue by ultrasound. While the focal size is larger than ultrasound of higher frequencies, the focal size remains applicable for targets in the brain including the amygdala, which is around 1 cubic centimeter in volume or glioblastoma tumors, which can reach dozens of cubic centimeters.

The majority of studies that applied ultrasound to PFC-based nanoparticles used PFCs with boiling point below the body temperature. Although the Laplace pressure of sub-micron particles can effectively increase the boiling point, PFCs with low boiling points may suffer from instability issues, which may raise safety concerns. These data show that PFP-based nanoparticles not exposed to ultrasound spontaneously increased in size over time, whereas PFOB nanoparticles were stable (FIG. 5A). These data suggest that PFP-based nanoparticles should be used near-immediately following their production and that their time in the blood stream should be minimized, if possible. Further, low boiling point PFCs can form persistent microbubbles after vaporization by ultrasound. In fact, with respect to PFC safety, the FDA has thus far approved PFCs with a high boiling point for large-scale use in humans—PFOB in particular. Specifically, PFOB has been used in liter-quantities in humans as agents that deliver oxygen into tissues. The PFOB-based products have included LiquiVent—an oxygen-carrying liquid drug, and Oxygent (both Alliance Pharmaceutical Corporation, San Diego, Calif., USA)—a blood substitution agent. The finding that PFOB can be used for effective release from nanoparticles at low ultrasound frequencies (FIG. 3, FIG. 4) should mitigate the boiling-point associated concerns. PFOB-based nanoparticles administered to two non-human primates and three rats (FIG. 6) and caused no detectable changes in vital signs.

The pharmacokinetics of blood clearance of the PFOB-based nanoparticles were evaluated in two macaque monkeys. The nanoparticles showed a rapid initial decay with half-life of 3.1 min in the primate and a later phase decay with half-life 195 minutes (FIG. 7). This is on the order of the values reported in other studies that used PEGylated nanoparticles. The half-life can be controlled by the type and chain length of the copolymers constituting the shell. Various copolymer shells, and PEG-based copolymers in particular, have been approved by the FDA for a variety of indications.

The monkey blood clearance data were captured with two decaying exponentials (FIG. 7). The dual exponential decay was similar to that reported using PFP-based particles in rats, 15 and suggests an involvement of two distinct clearance processes or organs. In a marmoset, two macaques, and three rats, PFOB-based nanoparticles were primarily degraded in the liver, with notable traces also observed in the kidneys (FIG. 8). This finding aligns with those established using PFC-based nanoparticles in rats. Whether these two organs could produce the dual nature of the clearance kinetics observed in FIG. 7 should be evaluated in future studies. Lungs may also contribute to the clearance. In the liter quantities introduced into humans, PFOB can be eliminated from the body in large part through exhalation.

The in vitro release data presented here contribute to the understanding of the release mechanism. Thus far, the predominant hypothesis of action has been the vaporization of the PFC droplet upon the impact of focused ultrasound. In this mechanism, the thermal and mechanical aspects of propagating ultrasound exceed the vaporization threshold governed by the PFC boiling point and the Laplace pressure. The PFC core increases in size (up to a 5-fold increase in diameter), which contributes, in addition to any superimposed effects of ultrasound, to the drug release. Indeed, these data at the common range of ultrasound frequencies, 900 kHz, provide two lines of evidence supporting this hypothesis. First, the release increases with decreasing boiling point (FIG. 3B), as expected from the vaporization hypothesis. Second, thermal energy delivered by ultrasound is known to be proportional to pressure squared. A quadratic dependence of the release on pressure was discovered (FIG. 4B).

However, these results (FIG. 3A) suggest that ultrasound of frequencies lower than those in the common diagnostic range may mediate more effective drug release. Lower frequencies are known to accentuate mechanical effects, which can take two forms. First, ultrasound can induce cavitation, the formation of gaseous nuclei from dissolved gasses under the low-pressure epochs of the ultrasound pressure wave. Cavitation can provide useful mechanical forces until it exceeds a threshold at which the formed gaseous nuclei collapse and cause damage. From the FDA's 510(k) Track 3 standpoint, cavitation is unlikely to occur for mechanical index—defined as ultrasound pressure divided by the square root of frequency—values below 1.9. A 300 kHz, 1.0

MPa pressure at target yields a mechanical index of 1.83. Despite being below the value of 1.9, this pressure level already drives significant release (FIG. 4A). Thus, if cavitation is involved, it is either in the safe range or just one of several mechanisms. Second, PFCs are known to strongly bind oxygen. The negative pressure of the ultrasonic wave can produce an outward motion of the dissolved gas. The resulting forces are likely to mechanically distort the shell of the nanoparticle on a cycle-by-cycle basis and thus perpetuate release. Whether mediated by oxygen or other molecules, the maximal displacement of a particle in the ultrasound path, is linearly proportional to the ultrasound pressure amplitude Embedded Image, where F is the ultrasound frequency and Z the acoustic impedance. Indeed, the 300 kHz data show a linear dependence of the release on pressure (FIG. 4A). This mechanism is supported by high-speed imaging, which did not detect a persistent phase change despite effective release in rodents. Together, these findings indicate that both thermal and mechanical effects can be at play, depending on the applied ultrasound frequency. Higher frequencies accentuate thermal effects (PFC vaporization), whereas lower frequencies accentuate mechanical effects (cavitation or cycle-by-cycle particle displacement).

In summary, nanoparticles filled with high-boiling point PFCs-PFOB in particular—can be effectively activated with ultrasound at low (300 kHz) frequencies. The effect is consistent with a mechanical expansion of the core. In addition, such nanoparticles can be safely injected into the bloodstream of primates, have a half-life on the order of dozens of minutes, and are degraded mainly in the liver. This study informs the use of specific PFC-based nanoparticles and ultrasound parameters for effective, safe, and targeted drug release in humans.

### Example 3

### Safety and Effectiveness of Local Drug Delivery

The effectiveness and safety of the local drug delivery was validated using propofol-filled PFOB nanoparticles into two non-human primates (NHPs) (FIG. 10A). A low-frequency (450 kHz), low-intensity (1 MPa at target) ultrasound stimulus was applied to release the drug within the left or the right lateral geniculate nucleus (LGN). The NHPs were engaged in a standard, stimulus onset asynchrony task in which they decided to look at a left or a right target, whichever appeared first. The release of the drug by the low-intensity, low-frequency ultrasound within the right or the left LGN modulated behavior in the expected and spatially-specific manner (FIG. 10B). The released propofol, which is a neuroinhibitory drug, biases the monkey's choice behavior in the expected, ipsilateral direction. The effect is significant during several epochs (black bars) already for the relatively low number of collected sessions (n=5 (n=4) sessions of propofol release in the right (left) LGN). The duration of the effect is consistent with the duration of the neuroinhibitory effects of propofol reported in the literature. The behavioral effects for propofol release in the left and right LGN point in the opposite direction (orange versus blue), which confirms the release specificity within the respective LGN. Moreover, no behavioral or physiological deficits were observed upon repeated administration of the nanoparticles; the animals performed normally the following day. To further evaluate safety, blood draws were obtained 1.5-hours after each injection and samples were analyzed using clinical chemistry and hematology tests. Blood analyses showed normal clinical chemistry and hematology values. Metrics relevant to liver and immune function are provided in Table 2. There was an increased white blood cell count 1.5-hours following the nanoparticle injection; nonetheless, the increase was within the normal range. Values relevant to the function of the liver and the immune system are shown in columns.

TABLE 2
Blood Analyses Relevant to Liver and Immune Functions Pre- and Post-Administration
of Propofol via PFOB Nanoparticles Activated by Ultrasound

| | | | | | | | |
|---|---|---|---|---|---|---|---|
| | | | | | Total | Total | |
| | ALP | AST | ALT | Albumin | Bilirubin | Protein | WBC |
| Date | (U/L) | (U/L) | (U/L) | (g/dL) | (mg/dL) | (g/dL) | (K/μL) |

Non-human primate 1

| | | | | | | | |
|---|---|---|---|---|---|---|---|
| Baseline (May 15, 2022) | 173 | 28 | 29 | 4.7 | 0.2 | 7.6 | 8.4 |
| 1.5 h post inj. (Aug. 3, 2022) | 171 | 21 | 53 | 4.3 | 0.2 | 7.2 | 14.1 |
| Prior to Inj. (Aug. 10, 2022) | 168 | 33 | 85 | 4.2 | 0.2 | 7.2 | 9.8 |
| 1.5 h post inj. (Aug. 10, 2022) | 171 | 29 | 85 | 4.4 | 0.2 | 7.5 | 14.2 |
| Prior to Inj. (Aug. 19, 2022) | 155 | 24 | 70 | 4.1 | 0.2 | 6.9 | 7.1 |
| 1.5 h post inj. (Aug. 19, 2022) | 157 | 24 | 72 | 4.2 | 0.2 | 7.1 | 14.5 |

Non-human primate 2

| | | | | | | | |
|---|---|---|---|---|---|---|---|
| Baseline (May 15, 2022) | 93 | 41 | 29 | 3.9 | 0.2 | 7.2 | 7.4 |
| Prior to Inj. (Sep. 14, 2022) | 64 | 52 | 127 | 4.1 | 0.2 | 7.4 | 10.9 |
| 1.5 h post inj. (Sep. 14, 2022) | 64 | 41 | 120 | 3.9 | 0.2 | 7.1 | 12.4 |
| Prior to Inj. (Sep. 16, 2022) | 61 | 33 | 68 | 3.8 | 0.2 | 7.2 | 6.7 |
| 1.5 h post inj. (Sep. 16, 2022) | 60 | 34 | 69 | 3.8 | 0.2 | 7.1 | 9.4 |
| Prior to Inj. (Oct. 5, 2022) | 57 | 27 | 72 | 62 | 3.8 | 0.2 | 7.1 |
| 1.5 h post inj. (Oct. 5, 2022) | 57 | 31 | 71 | 87 | 3.8 | 0.2 | 7.0 |

### Example 4

### Encapsulation of Additional Active Pharmaceutical Agents

Mycophenolate mofetil was encapsulated into microparticles using the same method as for propofol as described above.

The encapsulation of ketamine used a slightly modified procedure, in which ketamine was encapsulated directly in the starting micelles before the conversion of the micelles to nanoparticles. Ketamine is normally available in the salt form, ketamine hydrochloride. This was converted into the hydrophobic free base form by adding 3 M NaOH to a solution of ketamine hydrochloride.

### Example 5

Drug Release of Propofol, Mycophenolate Mofetil, and Ketamine from Nanoparticles

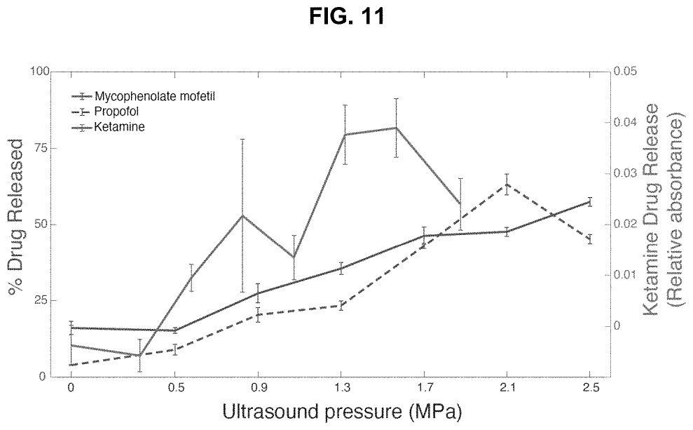

The release of propofol, mycophenolate mofetil, and ketamine from nanoparticles was performed as described above. The release was measured as a function of ultrasound pressure. See FIG. 11. Propofol and mycophenolate mofetil release was quantified by UV/visible spectroscopy and ketamine release was quantified by UV/visible fluorescence.

### Example 6

### Nanoparticle Imaging

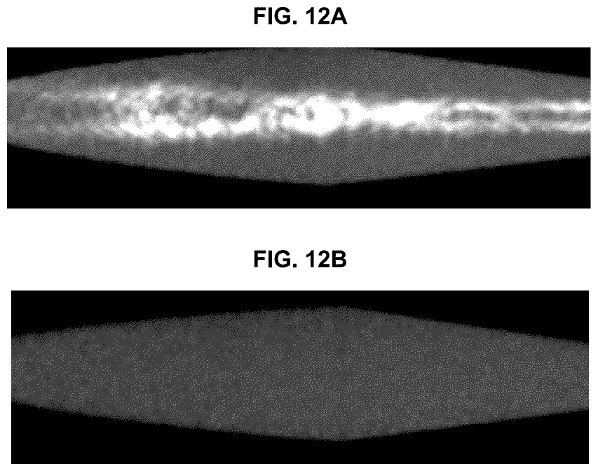

Nanoparticles with and without PFOB were prepared as described herein. The nanoparticles were imaged with computerized tomography (CT) using a common CT scanner (Multi-Modality CT, Inveon). Nanoparticles containing PFOB were visible under CT, whereas those without PFOB were not visible. See FIG. 12A-B. The contrast in the PFOB nanoparticles is due to bromide in the PFOB molecule.

1. An ultrasound release-inducible nanoparticle composition, comprising:
a nanoparticle core comprising perfluorooctylbromide (PFOB);
a copolymer matrix shell surrounding the nanoparticle core; and
one or more pharmaceutical agents.

2. The composition of claim 1, wherein the one or more pharmaceutical agents comprise propofol, ketamine, or mycophenolate mofetil.

3. The composition of claim 1, wherein the copolymer matrix shell comprises poly(ethylene glycol)-β-poly(D,L-lactide) (PEG:PDLLA) block copolymer.

4. The composition of claim 1, wherein the ultrasound release-inducible nanoparticle composition is configured to release the one or more pharmaceutical agents from the nanoparticle composition upon application of ultrasound.

5. The composition of claim 4, wherein the ultrasound frequency comprises about 100 kHz to about 650 kHz.

6. The composition of claim 5, wherein the low intensity ultrasound frequency comprises about 300 kHz.

7. The composition of claim 4, wherein the ultrasound release-inducible nanoparticle composition is configured to release the one or more pharmaceutical agents from the nanoparticle composition upon application of an ultrasound pressure ranging from about 1 MPa to about 3 MPa.

8. The composition of claim 7, wherein the ultrasound pressure comprises about 2 MPa.

9. The composition of claim 1, wherein the ultrasound release-inducible nanoparticle composition has a boiling point of up to about 142° C.

10. The composition of claim 1, wherein the ultrasound release-inducible nanoparticle composition has a diameter size ranging from about 300 nm to about 900 nm.

11. A method of releasing or activating one or more pharmaceutical agents from an ultrasound release-inducible nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents; and
administering ultrasound to the ultrasound release-inducible nanoparticle composition to release or activate the one or more pharmaceutical agents.

12. The method of claim 11, wherein the one or more pharmaceutical agents comprise propofol, ketamine, or mycophenolate mofetil.

13. The method of claim 11, wherein the copolymer matrix shell comprises poly(ethylene glycol)-β-poly(D,L-lactide) (PEG:PDLLA) block copolymer.

14. The method of claim 11, wherein the ultrasound frequency comprises about 100 kHz to about 650 kHz.

15. The method of claim 14, wherein the low intensity ultrasound frequency comprises about 300 kHz.

16. A method of treating a subject suffering from a disease or disorder using an ultrasound release-inducible nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents;
administering the ultrasound release-inducible nanoparticle composition to the subject; and
administering ultrasound to the subject to release the one or more pharmaceutical agents from the ultrasound release-inducible nanoparticle composition.

17. The method of claim 16, wherein the one or more pharmaceutical agents comprise propofol, ketamine, or mycophenolate mofetil.

18. The method of claim 16, wherein the copolymer matrix shell comprises poly(ethylene glycol)-β-poly(D,L-lactide) (PEG:PDLLA) block copolymer.

19. The method of claim 16, wherein the ultrasound frequency comprises about 100 kHz to about 650 kHz.

20. The method of claim 19, wherein the low intensity ultrasound frequency comprises about 300 kHz.

21. A method of performing imaging in a subject using an ultrasound release-inducible nanoparticle composition, the method comprising:
preparing an ultrasound release-inducible nanoparticle composition comprising:
a nanoparticle core comprising perfluorooctylbromide (PFOB);
a copolymer matrix shell surrounding the nanoparticle core; and
one or more pharmaceutical agents;
administering the ultrasound release-inducible nanoparticle composition to the subject;
administering ultrasound to the subject to release the one or more pharmaceutical agents from the ultrasound release-inducible nanoparticle composition; and
performing imaging on the subject.

22. The method of claim 21, wherein the imaging comprises CT, MRI, ultrasound, or combinations thereof.

23. The method of claim 21, wherein the one or more pharmaceutical agents comprise propofol, ketamine, or mycophenolate mofetil.

24-28. (canceled)
